(19) United States
(12) Patent Application Publication
(10) Pub. No.: US 2005/0087193 A1
(43) Pub. Date: Apr. 28, 2005

(54) Female inserted enterance protection

(76) Inventor: Sylvia Scott, Philadelphia, PA (US)

Correspondence Address: Sylvia Scott, 222 W. Zeralda St., Philadelphia, PA, 19144, US

(21) Appl. No.: 10/690,879

(22) Filed: Oct. 23, 2003

(51) Int. Cl. A61F 6/04

(52) U.S. Cl. 128/844

(57) ABSTRACT

The protective female panty garment apparatus for releasable securing a female condom. This ladies Undergarment that would include condom has an elastic waistband and leg bands. In the design of this Cotton or other undergarment material the crotch would be made of latex and have a cylindrical Extension that would serve as the condom during sexual activity. This condom would be inserted into the Woman's Vagina prior to intercourse. The undergarment can be worn all day without any maintenance This undergarment is made to provide a barrier to the entrance of male sperm and bodily fluids which May cause pregnancy or spreading of diseases such as AIDS.

### REFERENCES CITED

| | | | |
|---|---|---|---|
| 5,596,997 | Max M. Ababi | Jan. 28, 1997 | 128/844 |
| 5,687,741 | Karin S. Torger | Nov. 18, 1997 | 128/842 |
| 5,515,862 | Elie Artsi | May 14, 1996 | 128/830 |
| 5,535,757 | Andrew Fleming Jr. | Jul. 16, 1996 | 128/842 |
| 6,341,607 | Chantal Couvreur | Jan. 29, 2002 | 128/844 |

### FIELD OF THE INVENTION

The Present invention relates to having protection from Sexual Transmitted Transmitted Disease during sexual activity. This undergarment is easy to use and handle before and after sexual intercourse. This contraceptive device offers protect against AIDS and other life threatening diseases. Although there are many different types and method of contraception we find the Panty Condom a method of having protection when a simultaneous or spontaneous relationship occur. The most common method for prevention of AIDS is the use of condoms. When a Male uses a condom the Female uncovered areas become vulnerable. Females can only wear this Panty Protection for women. Needs for this type of prophylactic device, which is designed to be conformable and can be worn all day just like any other panty or undergarment for extended period of time. In crotch area a cylindrical design of Silicone Rubber will serve as the Condom during sexual activity. Just like in any under garment various embodiments of prophylactics and other materials will have arrange of sizes and lengths. The pouch will also range of sizes and lengths. The Pouch and Flange are made of Silicone Rubber. Adhesive is used to securely affix the Garment to the Flange. Although several different Adhesives or Adhesive Tape can be used on this Invention. The Adhesive must be able to not irritate the human skin in any way.

### DESCRIPTION OF THE DRAWINGS

FIG. 1 is a perspective view of the Front Embodiment of the present invention.

FIG. 2 is a Perspective view of the Back Embodiment of the present invention.

FIG. 3 is an outside view of the Condom in its sealed position.

FIG. 4 is an cross section of the Condom to show the layers of the design that makes the Panty Condom.

FIG. 5 is the Top view of the Embodiment of the present invention. The Top view show the inside of the Panty Condom that would be in contact with the Female.

### DETAIL DESCRIPTION OF THE DRAWINGS

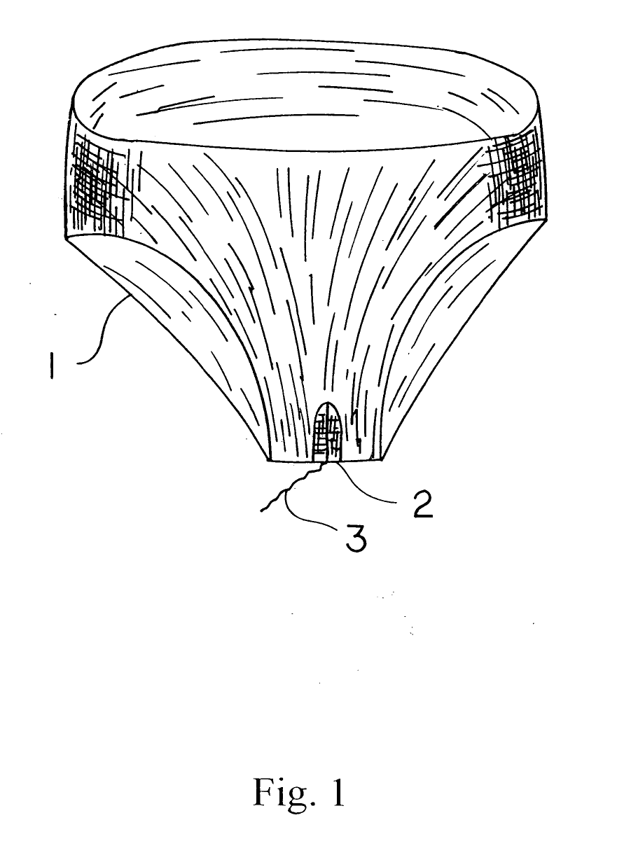

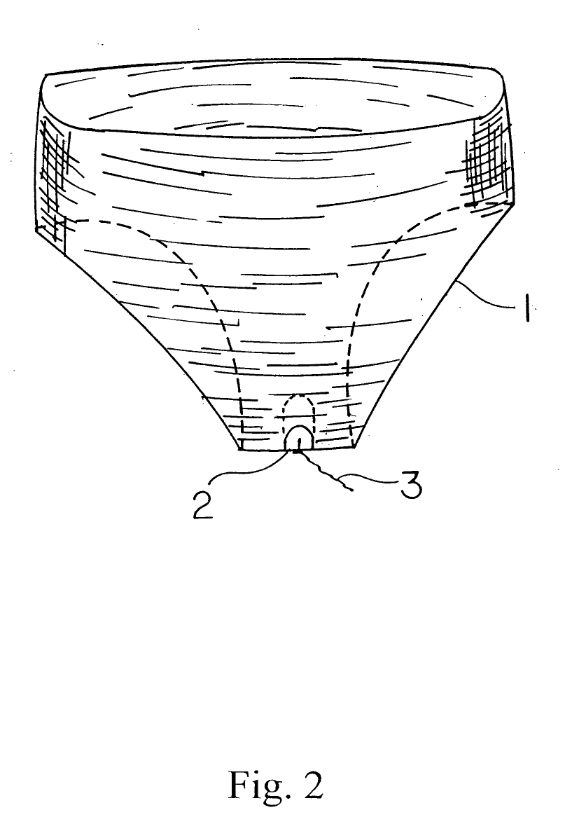

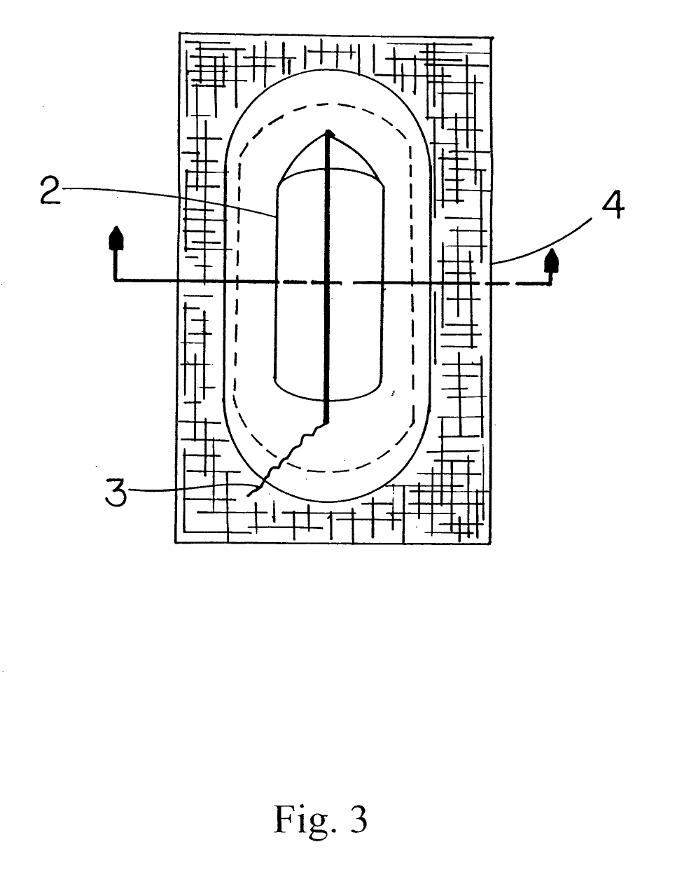

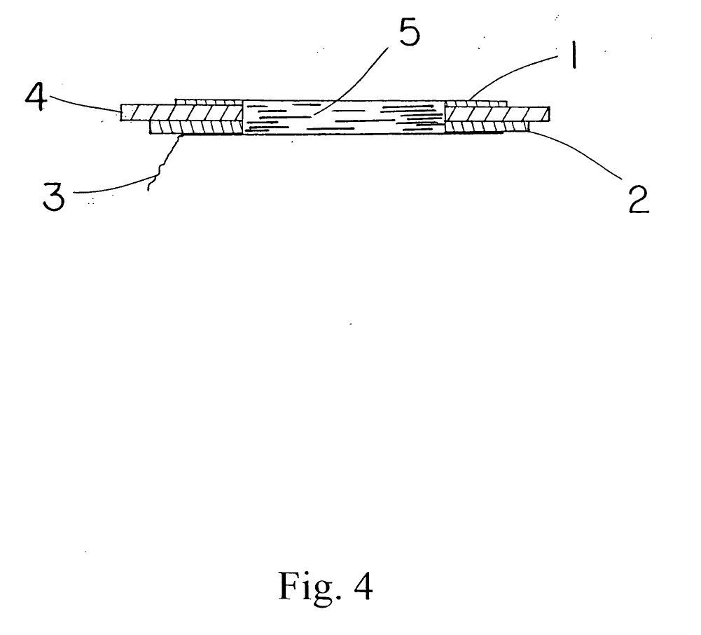

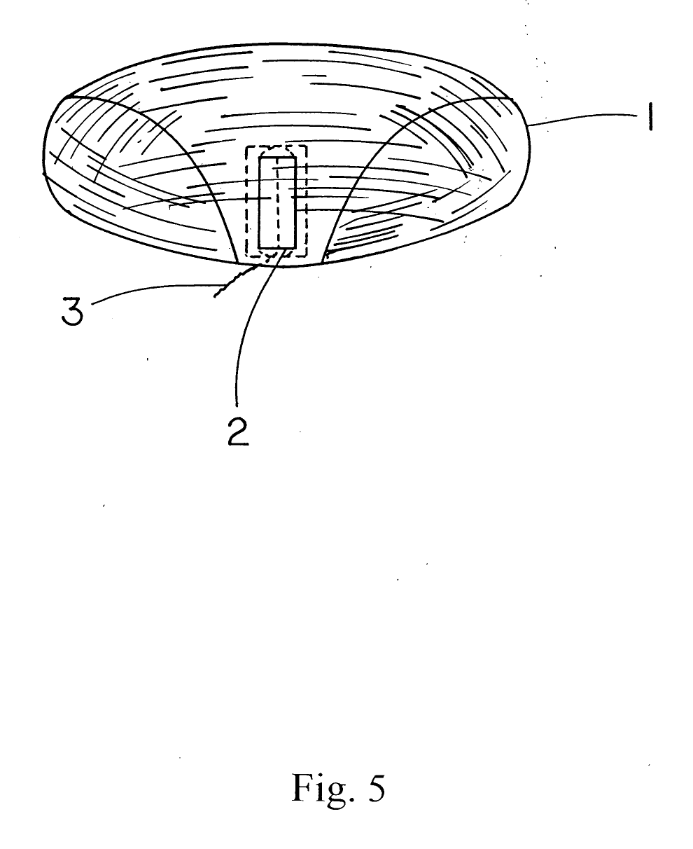

The present invention is designed for a woman to be prepared in advance prior to having sexual intercourse. Referring to FIG. 1, Panty 1 is a front view of this article and shows the Panties perspective before wearing by the female. Referring to FIG. 1, The Panty Condom is shown in perspective and the Condom is heat sealed between the outer and inters Panty Fabric and can never be disassembled with out destroying the Panty garment from its original manufactured design. Referring to FIG. 1 .the String 3 is shown in the Front view because it may hang a small length to help the female find the String when needed. Referring to FIG. 1, the String 3 is also connected to the Condom. No. 2 in FIG. 1, is sealed into slot position until the String is pulled from the Silicone Rubber that is used to make the Condom No. 3 holds its closed position. Referring to FIG. 2 is the Back view of the Panty No. 1, of this Panty Garment. Referring to FIG. 3 No. 2 is the Condom, which is formed out of Silicone Rubber to the size needed. Referring to the String No. 3 Shows the layout of the String No. 3 in relation with the sealing of the Condom No. 2 in FIG. 3. As shown in FIG. 3 the Flange No. 4 is designed as part of the Condom No. 2 in FIG. 3 to support to mounting of this invention. The Flange No. 4 in FIG. 3 has enough Silicone Rubber edge to fully support the Condom No. 2 in FIG. 3 and not cause discomfort to the female is wearing the Panty Garment. In Referring to FIG. No. 4 is a Cross Section of the Flange Reference No. 4. This view of the drawing in FIG. 4 is to show all the pieces of this Panty Condom. The Panty Condom pieces are assembled with a Heating method of the Silicone Rubber which is used to mount to the inter and outer layer of the Panty textile No. 1 in FIG. 4. The Condom No. 2 in FIG. 4 is attach this and keep its position with the Panty Fabric, No. 1 in FIG. 4. These parts No. 1, 2,4 in FIG. 4 are sealed for the life of the Panty Garment. Referring to No. 5 in FIG. 4 Shows the opening section of the Condom No. 2 which is part of No. 4 in FIG. 4. This open section No. 5 in FIG. 4 only opens when the String No. 3 is pulled and the seal of the Condom No. 2 in FIG. 4 will allow this invention to protect its user. In FIG. 5 is a Top view of the Panty Garment and shows looking down at the Panty's inside. The dotted line shows the Flange No. 4 in FIG. 5. being mounted on to the Panty Fabric No. 1 in FIG. 5. Referring to No. 3 the String in FIG. 5 shows the availability of releasing the the Condom No. 2 when needed. All the views show in this invention help describe this Panty Condom as a safety garment for the protection of women and men.

1. A combination undergarment and condom protection.

2. I claim of having a disposable Panty with Condom protection.

3. I claim the condom of having a Flange design being original and is all of my own concept.

4. I claim the String that opens the Condom from its sealed position is my own concept and being original.
